(19) United States
(12) Patent Application Publication
(10) Pub. No.: US 2008/0027092 A1
(43) Pub. Date: Jan. 31, 2008

(54) 1-Acetic Acid-Indole, -Indazole and -Benzimidazole Derivatives Useful for the Treatment of Respiratory Disorders

(76) Inventors: Roger Victor Bonnert, Leicestershire (GB); Rukhsana Tasneem Mohammed, Leicestershire (GB); Simon Teague, Leicestershire (GB)

Correspondence Address: FISH & RICHARDSON P.C., P.O BOX 1022, MINNEAPOLIS, MN, 55440-1022, US

(21) Appl. No.: 10/580,576

(22) PCT Filed: Nov. 24, 2004
(86) PCT No.: PCT/GB04/04937

§ 371 Date: May 25, 2007

(30) Foreign Application Priority Data
Nov. 26, 2003 (SE) 0303180-4

(51) Int. Cl. A61K 31/4709; A61K 31/416; A61P 11/00; C07D 231/56; C07D 471/06; C07D 401/04; A61P 11/06; A61K 31/435

(52) U.S. Cl. 514/300; 514/314; 514/403; 546/113; 546/167; 548/361.5

(57) ABSTRACT

The present invention relates to substituted indoles of formula (I) useful as pharmaceutical compounds for treating respiratory disorders.

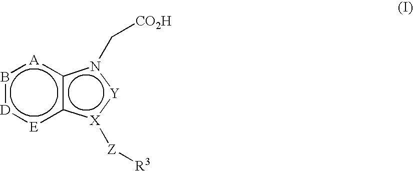

The present invention relates to substituted heterocycles useful as pharmaceutical compounds for treating respiratory disorders, pharmaceutical compositions containing them, and processes for their preparation.

EPA 1 170 594 discloses methods for the identification of compounds useful for the treatment of disease states mediated by prostaglandin D2, a ligand for orphan receptor CRTH2. GB 1356834 discloses a series of compounds said to possess anti-inflammatory, analgesic and antipyretic activity. It has now surprisingly been found that certain indazole acetic acids are active at the CRTH2 receptor, and as a consequence are expected to be potentially useful for the treatment of various respiratory diseases, including asthma and COPD.

In a first aspect the invention therefore provides a compound of formula (I) or a pharmaceutically acceptable salt thereof:

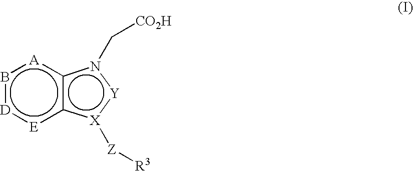

in which
each of A, B, D and E is independently C—R¹ or N;

### Y═C—R², N or C═O;

### Z is oxygen, sulphur, a C₁₋₆alkylene chain or a bond;

R¹ is independently selected from hydrogen, halogen, CN, nitro, S(O)ₓR⁶, OR⁶, SO₂NR⁴R⁵, CONR⁴R⁵, NR⁴R⁵, NR⁷SO₂R⁷, NR⁷C(O)ₓR⁷, C₂-C₆ alkenyl, C₂-C₆ alkynyl, C₁-₆alkyl, aryl or heteoroaryl, the latter five groups being optionally substituted by one or more substituents independently selected from 1-3 halogen atoms, —OR⁷ and —NR⁴R⁵, S(O)xR⁸, C(O)NR⁴R⁵, where x is 0,1 or 2;

R² is C₁₋₆alkyl which may be optionally substituted by one or more substituents independently selected from halogen atoms, aryl, —OR⁹ and —NR¹⁰R¹¹;

R³ is an aryl or heteroaryl group each of which is optionally substituted by one or more substituents independently selected from halogen, CN, nitro, S(O)ₓR⁶, OR⁷, SO₂NR⁴R⁵, CONR⁴R⁵, NR⁴R⁵, NR⁷SO₂R³, NR⁷C(O)ₓR⁶, C₂-C₆ alkenyl, C₂-C₆ alkynyl, C₁₋₆ alkyl, the latter three groups being optionally substituted by one or more substituents independently selected from halogen atoms, —OR⁶ and —NR⁴R⁵, where x=0,1 or 2;

R⁴ and R⁵ independently represent a hydrogen atom, a C₁₋₆alkyl group, or aryl group the latter two of which may be optionally substituted by one or more substituent groups independently selected from halogen atoms, aryl, —OR¹² and —NR¹³R¹⁴, —CONR¹³R¹⁴, —NR¹³COR¹⁴, —SO₂NR¹³R¹⁴, NR¹³SO₂R¹⁴; or

R⁴ and R⁵ together with the nitrogen atom to which they are attached can form a 3-8 membered saturated heterocylic ring optionally containing one or more atoms selected from O, S, NR¹⁵, and itself optionally substituted by C₁₋₃ alkyl, halogen;

R⁶ represents a C₁₋₆alkyl which may be optionally substituted by one or more substituents independently selected from halogen atoms, aryl, —OR⁹ and —NR¹⁰R¹¹.

each of R⁷, R⁸ R⁹, R¹⁰, R¹¹, R¹², R¹³, R¹⁴, independently represents a hydrogen atom, C₁-C₆, alkyl, an aryl or a heteroaryl group which may be optionally substituted by one or more halogen atoms, OH, O—C₁-C₆alkyl; and

R¹⁵ is hydrogen, C₁₋₄ alkyl, —COC₁-C₄ alkyl, —COQC₁-C₄alkyl, Q=O or NR⁶, provided that:

the number of nitrogen atoms within the ring ABDE is 1 or 2 when Y is CR²

R³ cannot be phenyl when Y is C═O and X is nitrogen.

In the context of the present specification, unless otherwise indicated, an alkyl or alkenyl group or an alkyl or alkenyl moiety may be linear, branched or cyclic.

Aryl is phenyl and naphthyl. Heteroaryl is defined as a 5-7 membered aromatic ring or can be 6,6- or 6,5-fused bicyclic each ring containing one or more heteroatoms selected from N, S and O. Examples include pyridine, pyrimidine, thiazole, oxazole, pyrazole, imidazole, furan, isoxazole, pyrrole, isothiazole and azulene, naphthyl, indene, quinoline, isoquinoline, indole, indolizine, benzo[b]furan, benzo[b]thiophene, 1H-indazole, benzimidazole, benzthiazole, benzoxazole, purine, 4H-quinolizine, cinnoline, phthalazine, quinazoline, quinoxaline, 1,8-naphthyridine, pteridine, quinolone.

Heterocyclic rings as defined for R⁴ and R⁵ means saturated heterocycles, examples include morpholine, thiomorpholine, azetidine, imidazolidine, pyrrolidine, piperidine and piperazine.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The compound of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate. Preferred salts include sodium salts.

The term alkyl, whether alone or as part of another group, includes straight chain, branched and cyclic alkyl groups.

Preferably, when Y is nitrogen or C—R², Z is a bond or sulfur and R³ is aryl or heteroaryl.

Preferably R¹ is hydrogen, alkyl, substituted alkyl, halogen or nitrile, NR⁷SO₂R⁷, NR⁷C(O)ₓR⁷.

Most preferably R¹ is hydrogen, phenyl, CF₃, CN, alkyl or halogen, more preferably hydrogen, phenyl, CF₃, CN, methyl, iodo or chloro.

The substituent(s) R¹ can be present at any position of the ring ABDE, more preferably the C—R¹ group is present at positions D and (or) E.

Preferably, when the ring ABDE contains a nitrogen atom, it can be present at any of the four positions ABDE, more preferably the N atoms are present at positions A, D or E.

Preferably the number of nitrogen in ring ABDE is 1-2 when Y is CR², more preferably the number of nitrogen atoms is 1 when Y is CR².

Preferably when Y is N or C═O, the number of nitrogen atoms within the ring ABDE is 0-2. More preferably when Y is N or C═O, the number of nitrogen atoms within the ring is 1, that is either A, D or E is N.

More preferably when Y is N or C═O the number of nitrogen atoms contained in ring ABDE is 0.

Preferably when Y is C═O, X is nitrogen and Z is a bond.

Preferably when Y is nitrogen, or C—R², X is carbon, Z can be oxygen, sulfur, methylene or a bond, preferably sulfur, methylene or a bond. Preferably R² is alkyl, more preferably methyl.

Examples of generic structure types (I) are:

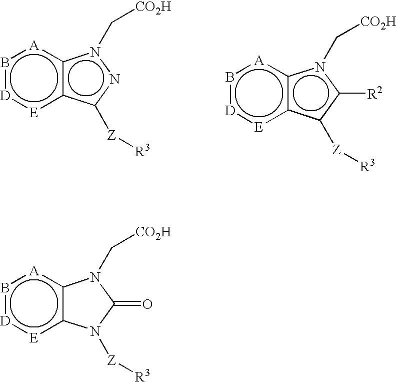

Suitably R³ is aryl or heteroaryl. Suitable heteroaryl groups includes a 6,6- or 6,5-fused bicyclic aromatic ring optionally containing one to three heteroatoms selected from nitrogen, oxygen or sulphur, or a 5- to 7-membered heterocyclic ring containing one to three heteroatoms selected from nitrogen, oxygen or sulphur.

Examples of 6,6- or 6,5-fused bicyclic aromatic rings include naphthyl, indene, quinoline, isoquinoline, indole, indolizine, benzo[b]furan, benzo[b]thiophene, 1H-indazole, benzimidazole, benzthiazole, purine, 4H-quinolizine, cinnoline, phthalazine, quinazoline, quinoxaline, 1,8-naphthyridine, pteridine, quinolone.

Examples of 5- to 7-membered heterocyclic rings include pyridine, pyrimidine, thiazole, oxazole, isoxazole, pyrazole, imidazole, furan, thiophene, pyrrole, isothiazole and azulene.

Preferably R³ is quinoline or phenyl, both optionally substituted as defined above. Substituents can be present on any suitable position of an R³ group, including nitrogen atoms where these are present. Preferred substituents for R³ groups include halogen, S(O)ₓR⁶, more preferably fluoro, chloro or SO₂Me.

Preferred compounds of the invention include:
5-methyl-3-(4-quinolinyl)-1H-indazole-1-acetic acid;
5-cyano-3-(4-quinolinyl)-1H-indazole-1-acetic acid;
3-(6-fluoro-4-quinolinyl)-4-(trifluoromethyl)-1H-indazole-1-acetic acid;
4-iodo-3-(4-quinolinyl)-1H-indazole-1-acetic acid;
3-[(4-chlorophenyl)thio]-5-iodo-1H-indazole-1-acetic acid;
3-(7-chloro-4-quinolinyl)-2-methyl-1H-pyrrolo[2,3-b]pyridine-1-acetic acid, sodium salt;
3-[(4-Chloro-2,4-cyclohexadien-1-yl)thio]-2,5-dimethyl-1H-pyrrolo[3,2-b]pyridine-1-acetic acid;
2,5-Dimethyl-3-[[4-(methylsulfonyl)-2,4-cyclohexadien-1-yl]methyl]-1H-pyrrolo[3,2-b]pyridine-1-acetic acid;
2,5-Dimethyl-3-[[4-(methylsulfonyl)phenyl]thio]-1H-pyrrolo[3,2-b]pyridine-1-acetic acid;
4-Chloro-3-[(4-chlorophenyl)thio]-2-methyl-1H-pyrrolo[3,2-c]pyridine-1-acetic acid;
4-Chloro-2-methyl-3-[[4-(methylsulfonyl)phenyl]thio]-1H-pyrrolo[3,2-c]pyridine-1-acetic acid;
3-[(4-Chlorophenyl)thio]-2-methyl-4-phenyl-1H-pyrrolo[3,2-c]pyridine-1-acetic acid;
2-Methyl-3-[[4-(methylsulfonyl)phenyl]thio]-4-phenyl-1H-pyrrolo[3,2-c] pyridine-1-acetic acid;
   and pharmaceutically acceptable salts thereof.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The compounds of formula (I) above may be converted to a pharmaceutically acceptable salt or solvate thereof, preferably a basic addition salt such as sodium, potassium, calcium, aluminium, lithium, magnesium, zinc, benzathine, chloroprocaine, choline, diethanolamine, ethanolamine, ethyldiamine, meglumine, tromethamine or procaine, or an acid addition salt such as a hydrochloride, hydrobromide, phosphate, acetate, fumarate, maleate, tartrate, citrate, oxalate, methanesulphonate or p-toluenesulphonate. Preferred salts include sodium salts.

It will be appreciated that certain functional groups may need to be protected using standard protecting groups. The protection and deprotection of functional groups is for example, described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

Compounds of formula (I) can be prepared by reaction of a compound of formula (II):

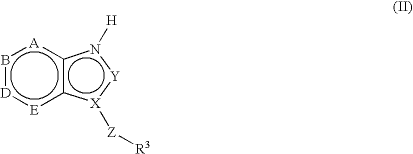

in which A, B, D, E, Y, Z and R³ are as defined in formula (I) or are protected derivatives thereof, with a compound of formula (III):

L-CH₂CO₂R¹⁶

where R¹⁶ is an alkyl group and L is a leaving group in the presence of a base, and optionally thereafter in any order:

removing any protecting group

hydrolysing the ester group R¹¹ to the corresponding acid

forming a pharmaceutically acceptable salt.

The reaction can be carried out in a suitable solvent such as THF using a base such as sodium hydride or the like. Suitable groups R¹⁶ include C₁₋₆ alkyl groups such as methyl, ethyl, or tertiary butyl. Suitable L is a leaving group such as halo, in particular bromo. Preferably the compound of formula (III) is methyl, ethyl or t-butyl bromoacetate.

Hydrolysis of the ester group R¹⁶ can be carried out using routine procedures, for example by stirring with aqueous sodium hydroxide or trifluoroacetic acid.

Compounds of formula (II in which Y is N, can be prepared by reaction of a compound of formula (IV):

Compounds of formula (IV) can be prepared by reacting a compound of formula (V) with hydrazine and heating at 100° C.

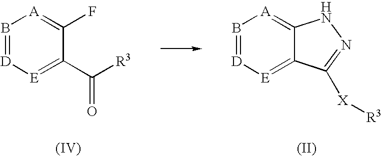

Compounds of formula (V) can be prepared by oxidation of a compound of formula (V). Suitable oxidation conditions are Swern or Dess-martin.

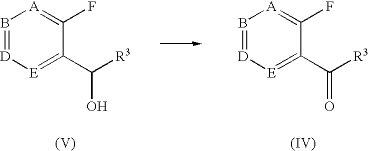

Compounds of formula (V) can be prepared by reacting a compound of formula (VI) with a compound of formula (VII)

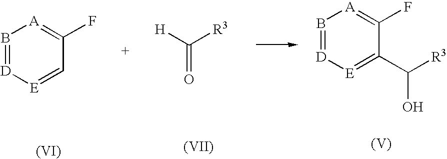

in which R³ is as defined in formula (II).

Compounds of formula (VII) and (VIII) are commercially available or can be prepared using standard chemistry well known in the art.

Certain compounds of formula (II) where Y═C—R² are prepared by reaction of compounds of formulae (IX) with compounds of formula (XI) using a procedure described in WO 01/07436.

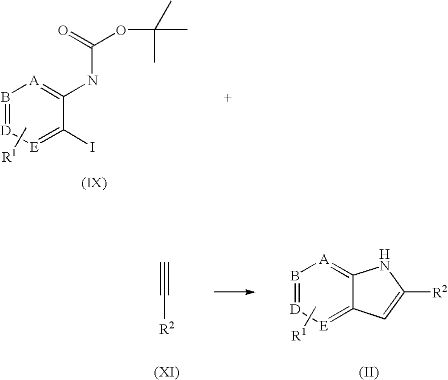

When Z is sulfur a compound of formula (II) is treated with a disulfide (Ixa) in the presence of a base such as potassium tertiary butoxide in a suitable sovent such as DMF or tertiary butanol and heating.

R³—S—S—R³ (IXa)

When Z is CH₂, the compound of formula (II) is treated with a Grignard reagen such as ethyl magnesium bromide and BrCH₂R³.

Compounds of formula (II) where Y═C═O and X═N, are synthesised from compounds of formula (XII) by treating with diphenylphosphoryl azide in triethylamine and a suitable solvent such as DMF.

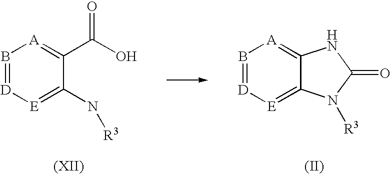

Compounds of formula (XII) can be prepared by reacting compounds of formula (XIII) and (XIV)

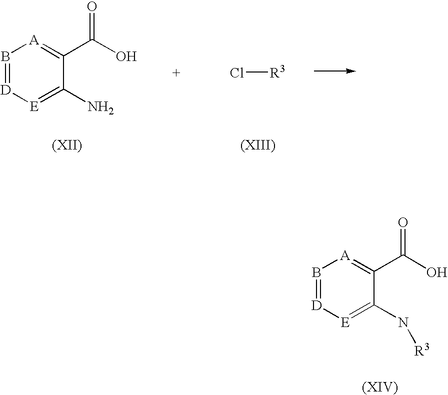

Certain compounds of formulae (II) (V) and (IV) or protected derivatives thereof are believed to be novel and form a further aspect of the invention.

It will be appreciated by those skilled in the art that in the processes of the present invention certain functional groups in the starting reagents or intermediate compound may need to be protected by protecting groups. Thus, the preparation of the compound of formula (I) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 3rd edition, T. W. Greene & P. G. M. Wuts, Wiley-Interscience (1999).

In a further aspect, the present invention provides the use of a compound of formula (I), pharmaceutically acceptable salt or solvate thereof for use in therapy.

The compounds of formula (I) have activity as pharmaceuticals, in particular as modulators of CRTh2 receptor activity, and may be used in the treatment (therapeutic or prophylactic) of conditions/diseases in human and non-human animals which are exacerbated or caused by excessive or unregulated production of PGD₂ and its metabolites. Examples of such conditions/diseases include:

In a further aspect, the present invention provides the use of a compound of formula (I), a prodrug, pharmaceutically acceptable salt or solvate thereof for use in therapy.

The compounds of formula (I) have activity as pharmaceuticals, in particular as modulators of CRTh2 receptor activity, and may be used in the treatment (therapeutic or prophylactic) of conditions/diseases in human and non-human animals which are exacerbated or caused by excessive or unregulated production of PGD₂ and its metabolites. Examples of such conditions/diseases include:

A compound of the invention, or a pharmaceutically acceptable salt thereof, can be used in the treatment of:
(1) (respiratory tract) obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) and adenovirus.
(2) (bone and joints) arthritides associated with or including osteoarthritis/osteoarthrosis, both primary and secondary to e.g. congenital hip dysplasia; cervical and lumbar spondylitis, and low back and neck pain; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies.
(3) (skin) psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions.
(4) (eyes) blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial.
(5) (gastrointestinal tract) glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema).
(6) (abdominal) hepatitis, including autoimmune, alcoholic and viral; fibrosis and cirrhosis of the liver; cholecystitis; pancreatitis, both acute and chronic.
(7) (genitourinary) nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female).
(8) (Allograft rejection) acute and chronic following, for example, transplantation of kidney, heart, liver, lung, bone marrow, skin or cornea or following blood transfusion; or chronic graft versus host disease;
(9) (CNS) Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes.
(10) Other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome.
(11) Other disorders with an inflammatory or immunological component; including acquired immune deficiency syndrome (AIDS), leprosy, Sezary syndrome, and paraneoplastic syndromes.
(12) (Cardiovascular); atherosclerosis, affecting the coronary and peripheral circulate pericarditis; myocarditis, inflammatory and auto-immune cardiomyopathies including myocardial sarcoid; ischaemic reperfusion injuries; endocarditis, valvulitis, and aortitis including infective (e.g. syphilitic); vasculitides; disorders of the proximal and peripheral veins including phlebitis and thrombosis, including deep vein thrombosis and complications of varicose veins.
(13) (Oncology) treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes.
(14) Diseases associated with raised levels of PGD₂ or its metabolites.

Thus, the present invention provides a compound of formula (I), or a pharmaceutically-acceptable salt or solvate thereof, as hereinbefore defined for use in therapy.

Preferably the compounds of the invention are used to treat diseases in which the chemokine receptor belongs to the CRTh2 receptor subfamily.

Particular conditions which can be treated with the compounds of the invention are asthma, rhinitis and other diseases in which raised levels of PGD₂ or its metabolites. It is preferred that the compounds of the invention are used to treat asthma.

In a further aspect, the present invention provides the use of a compound of formula (I), or a pharmaceutically acceptable salt or solvate thereof, as hereinbefore defined in the manufacture of a medicament for use in therapy.

The invention further relates to combination therapies wherein a compound of formula (1) or a pharmaceutically acceptable salts, solvate or in vivo hydrolysable ester thereof, or a pharmaceutical composition or formulation comprising a compound of formula (1) is administered concurrently or sequentially with therapy and/or an agent for the treatment of any one of asthma, allergic rhinitis, cancer, COPD, rheumatoid arthritis, psoriasis, inflammatory bowel diseases, osteoarthritis or osteoporosis.

In particular, for the treatment of the inflammatory diseases rheumatoid arthritis, psoriasis, inflammatory bowel disease, COPD, asthma and allergic rhinitis the compounds of the invention may be combined with agents such as TNF-α inhibitors such as anti-TNF monoclonal antibodies (such as Remicade, CDP-870 and D.sub2.E.sub7.) and TNF receptor immunoglobulin molecules (such as Enbrel.reg.), non-selective COX-1/COX-2 inhibitors (such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin), COX-2 inhibitors (such as meloxicam, celecoxib, rofecoxib, valdecoxib and etoricoxib) low dose methotrexate, lefunomide; ciclesonide; hydroxychloroquine, d-penicillamine, auranofin or parenteral or oral gold.

The present invention still further relates to the combination of a compound of the invention together with a leukotriene biosynthesis inhibitor, 5-lipoxygenase (5-LO) inhibitor or 5-lipoxygenase activating protein (FLAP) antagonist such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of a compound of the invention together with a receptor antagonist for leukotrienes LTB.sub4., LTC.sub4., LTD.sub4., and LTE.sub4. selected from the group consisting of the phenothiazin-3-ones such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention together with a PDE4 inhibitor including inhibitors of the isoform PDE4D.

The present invention still further relates to the combination of a compound of the invention together with a antihistaminic H.sub1. receptor antagonists such as cetirizine, loratadine, desloratadine, fexofenadine, astemizole, azelastine, and chlorpheniramine.

The present invention still further relates to the combination of a compound of the invention together with a gastroprotective H.sub2. receptor antagonist.

The present invention still further relates to the combination of a compound of the invention together with an α.sub1.- and α.sub2.-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of a compound of the invention together with anticholinergic agents such as ipratropium bromide; tiotropium bromide; oxitropium bromide; pirenzepine; and telenzepine.

The present invention still further relates to the combination of a compound of the invention together with a β.sub1.- to β.sub4.-adrenoceptor agonists such as metaproterenol, isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of a compound of the invention together with an insulin-like growth factor type I (IGF-1) mimetic.

The present invention still further relates to the combination of a compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of a compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of a compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to the combination of a compound of the invention together with antiviral agents such as Viracept, AZT, aciclovir and famciclovir, and antisepsis compounds such as Valant.

The present invention still further relates to the combination of a compound of the invention together with cardiovascular agents such as calcium channel blockers, lipid lowering agents such as statins, fibrates, beta-blockers, Ace inhibitors, Angiotensin-2 receptor antagonists and platelet aggregation inhibitors.

The present invention still further relates to the combination of a compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metryfonate.

The present invention still further relates to the combination of a compound of the invention together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinin-B.sub1. - and B.sub2.-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFβ); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK.sub1. and NK.sub3. receptor antagonists selected from the group consisting of NKP-608C; SB-233412 (talnetant); and D-4418; (xx) elastase inhibitors selected from the group consisting of UT-77 and ZD-0892; (xxi) TNFα converting enzyme inhibitors (TACE); (xxii) induced nitric oxide synthase inhibitors (iNOS) or (xxiii) chemoattractant receptor-homologous molecule expressed on TH2 cells, (CRTH2 antagonists).

The compounds of the present invention may also be used in combination with osteoporosis agents such as roloxifene, droloxifene, lasofoxifene or fosomax and immunosuppressant agents such as FK-506, rapamycin, cyclosporine, azathioprine, and methotrexate.

The compounds of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compounds of the invention can also be used in combination with existing therapeutic agents for the treatment of cancer. Suitable agents to be used in combination include:

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) Agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);

(vi) vascular damaging agents such as Combretastatin A4 and compounds disclosed in International Patent Applications WO99/02166, WO00/40529, WO00/41669, WO01/92224, WO02/04434 and WO02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In a still further aspect, the present invention provides the use of a compound of formula (I), or a pharmaceutically acceptable salt or solvate thereof, as hereinbefore defined in the manufacture of a medicament for the treatment of human diseases or conditions in which modulation of CRTh2 receptor activity is beneficial.

In the context of the present specification, the term “therapy” also includes “prophylaxis” unless there are specific indications to the contrary. The terms “therapeutic” and “therapeutically” should be construed accordingly.

The invention still further provides a method of treating diseases mediated by PGD2 or its metabolites wherein the prostanoid binds to its receptor (especially CRTh2) receptor, which comprises administering to a patient a therapeutically effective amount of a compound of formula (I), or a pharmaceutically acceptable salt, solvate or prodrug thereof, as hereinbefore defined.

The invention also provides a method of treating an inflammatory disease, especially psoriasis, in a patient suffering from, or at risk of, said disease, which comprises administering to the patient a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

For the above-mentioned therapeutic uses the dosage administered will, of course, vary with the compound employed, the mode of administration, the treatment desired and the disorder indicated.

The compound of formula (I), prodrugs and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as herein before defined, in association with a pharmaceutically acceptable adjuvant, diluent or carrier.

The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Preferably the compound of the invention is administered orally.

The present invention also provides a pharmaceutical composition comprising a compound of formula (I), or a pharmaceutically acceptable salt or solvate thereof, as herein before defined, in association with a pharmaceutically acceptable adjuvant, diluent or carrier.

The invention will now be illustrated by the following non-limiting examples in which, unless stated otherwise:

i) The title and sub-titled compounds of the examples and methods were named using the ACD labs/name program (version 6.0) from Advanced Chemical Development Inc, Canada;

ii) Unless stated otherwise, reverse phase preparative HPLC was conducted using a Symmetry, NovaPak or Ex-Terra reverse phase silica column;

iii) Flash column chromatography refers to normal phase silica chromatography iv) Solvents were dried with MgSO₄ or Na₂SO₄

v) Evaporations were carried out by rotary evaporation in vacuo and work-up procedures were carried out after removal of residual solids such as drying agents by filtration;

vi) Unless otherwise stated, operations were carried out at ambient temperature, that is in the range 18-25° C. and under an atmosphere of an inert gas such as argon or nitrogen;

vii) Yields are given for illustration only and are not necessarily the maximum attainable;

viii) The structures of the end-products of the formula (1) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;

ix) Intermediates were not generally fully characterised and purity was assessed by thin layer chromatography (TLC), high-performance liquid chromatography (HPLC), mass spectrometry (MS), infra-red (IR) or NMR analysis;

x) Mass spectra (MS): generally only ions which indicate the parent mass are reported when given, ¹H NMR data is quoted in the form of delta values (δ) for major diagnostic protons, given in parts per million (ppm) relative to tetramethylsilane (TMS) as an internal standard;

xi) The following abbreviations are used:

EtOAc Ethylacetate

DMF N,N-Dimethyl formamide

NMP N-methylpyrrolidine

THF tetrahydrofuran

RT room temperature

TFA trifluoroacetic acid

### EXAMPLE 1

### 5-methyl-3-(4-quinolinyl)-1H-indazole-1-acetic acid

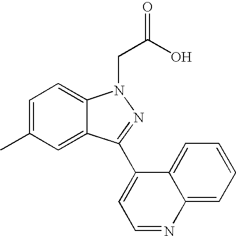

a) α-(2-fluoro-5-methylphenyl)-4-quinolinemethanol

n-BuLi (2.5M in hexanes, 5.1 ml) was added dropwise to 3-bromo-4-fluorotoluene (2 g) in THF (100 ml) at −78° C. The reaction mixture was stirred for 10 min and then 4-quinolinecarboxaldehyde (1.7 g) in THF (10 ml) was added dropwise and stirred for 40 min. The reaction mixture was quenched (water) allowed to reach RT and then extracted (EtOAc), dried (MgSO₄) and concentrated in vacuo. The residue was purified by chromatography (silica, eluting EtOAc:hexane; 6:4) to give the subtitle compound as a white solid (1.15 g).

MS ESI+ 267 [M+1]

b) (2-fluoro-5-methylphenyl)-4-quinolinyl-methanone

DMSO (1.26 ml) was added dropwise to a solution of oxalyl chloride (1.12 ml) in dichloromethane (40 ml) at −78° C. The solution was stirred for 30 min and then the product from step a (1.11 g) in dicloromethane (10 ml) was added dropwise. The reaction mixture was allowed to reach 0° C. over 2 h. Triethylamine (1.25 ml) was added keeping the temperature between (0-10° C.) and stirred for 5 min. The reaction mixture was quenched (water), separated the 2 layers. Then reextracted with dicloromethane. The combined organic extracts were dried (MgSO₄) and concentrated in vacuo. The residue was purified by chromatography (silica, eluting EtOAc:hexane; 1:1) to give the subtitle compound as a yellow solid (1.2 g).

MS ESI+ 265 [M+1]

c) 4-(5-methyl-1H-indazol-3-yl)-quinoline

Hydazine monohydrate (0.48 ml) was added to the product of step b) (0.52 g) in toluene (6 ml), and heated for 3 days at 110° C. The reaction was concentrated in vacuo and the residue was purified by chomatography (silica, eluting EtOAc:hexame 4:6), to give the subtitle compound (0.25 g).

MS ESI+ 260 [M+1]

d) 5-methyl-3-(4-quinolinyl)-1H-indazole-1-acetic acid, ethyl ester

NaH (60% dispersion in mineral oil, 50 mg) was added to the product from step c (245 mg) in THF (8 ml) under nitrogen. The reaction mixture was stirred for 10 min and then ethyl bromoacetate (0.11 ml) was added dropwise and the mixture stirred for a further 1 h. The reaction mixture was quenched with water, extracted (EtOAc). The organic phase was dried (MgSO₄) and concentrated in vacuo. The residue was purified by chromatography (silica, eluting EtOAc:hexame, 3:7), to give the subtitle compound (100 mg).

MS ESI+ 346 [M+1]

e) 5-methyl-3-(4-quinolinyl)-1H-indazole-1-acetic acid

The product from step b (70 mg) was treated with NaOH (2M, 0.2 ml), THF (1 ml) and methanol (1 ml). The resulting solution was stirred at room temperature for 3 h. The reaction mixture was concentrated in vacuo, then purified by reverse phase HPLC (eluting with ammonia and acetonitile), to give the title compound as a pale yellow solid (15 mg).

¹H NMR (DMSO) δ 2.43 (s, 3H), 5.38 (s,2H), 7.35 (d,1H), 7.5-7.83 (m,5H), 8.12 (d,1H), 8.53 (d, 1H), 9.03 (d, 1H).

MS APCI+ 318 [M+1]

### EXAMPLE 2

### 5-cyano-3-(4-quinolinyl)-1H-indazole-1-acetic acid

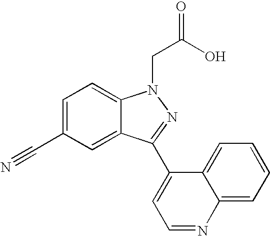

a) 4-fluoro-3-(hydroxy-4-quinolinylmethyl)-benzonitrile

The subtitle compound was prepared by the method of example 1 part a, using 2-fluro-4-cyanobromobenzene and quinoline-4-aldehyde.

¹H NMR (CDCl₃) δ 2.84 (d,1H), 6.85 (d,1H), 7.18-7.22 (m,1H), 7.52-7.89 (m,5H), 7.92 (d, 1H), 8.08 (d, 1H) and 8.97 (d,1H).

b) 4-fluoro-3-(4-quinolinylcarbonyl)-benzonitrile

The subtitle compound was prepared by the method of example 1 part b, using the product of step a).

¹H NMR (CDCl₃) δ 7.23-7.31 (m,1H), 7.39 (d,1H), 7.62 (t, 1H), 7.82 (t,1H), 7.84-7.98 (m,1H), 8.13-8.18 (m,2H), 8.2-8.25 (d, 1H), 9.05 (d,1H).

c) 3-(4-quinolinyl)-1H-indazole-5-carbonitrile

The subtitle compound was prepared by the method of example 1 part c, using the product of step b).

MS APCI+ 279 [M+1]

d) 5-cyano-3-(4-quinolinyl)-1H-indazole-1-acetic acid, ethyl ester

The subtitle compound was prepared by the method of example 1 part d, using the product of step c).

¹H NMR (CDCl₃) δ 0.83 (t,3H), 4.31 (q,2H), 5.34 (s,2H), 7.54-7.76 (m,4H), 7.8-7.84 (m,1H), 8.14 (s,1H), 8.27-8.31 (m, 2H), 9.08 (d,1H).

e) 5-cyano-3-(4-quinolinyl)-1H-indazole-1-acetic acid

The title compound was prepared by the method of example 1 part e) using the product of step d).

¹H NMR (DMSO) δ 5.16 (s,2H), 7.6-7.7 (m,1H), 7.78-7.96 (m,4H), 8.15-8.19 (d,1H), 8.42 (s, 1H), 8.78 (d,1H) and 9.05 (d,1H).

### EXAMPLE 3

### 3-(6-fluoro-4-quinolinyl)-4-(trifluoromethyl)-1H-indazole-1-acetic acid

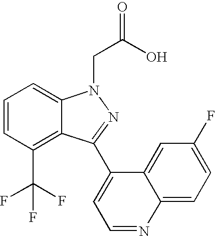

a) 7-fluoro-α-[2-fluoro-6-(trifluoromethyl)phenyl]-1-naphthalenemethanol

Prepared by the method of example 1 step a, using 2-bromo-1-fluoro-3-(trifluoromethyl)-benzene and 6-fluoroquinoline.

MS ESI+ 340 [M+1]

b) (6-fluoro-4-quinolinyl)[2-fluoro-6-(trifluoromethyl)phenyl]-methanone

Dess-martin periodinone (1.06 g) was added to the product of step a (0.85 g) in dichloromethane (25 ml). The solution was stirred for 2 h and then washed with sodium thiosulfate, sodium hydrogen carbonate and brine. The organic phase was dried (MgSO₄) and then concentrated in vacuo. The residue was purified by chomatography (silica, eluting EtOAC:hexame 3:7), to give the subtitle compound (420 mg).

MS ESI+ 338 [M+1]

c) 6-fluoro-4-[4-(trifluoromethyl)-1H-indazol-3-yl]-quinoline

The subtitle compound was prepared by the method of example 1 step c) from the product of step c)

MS ESI+ 314 [M+1]

d) 3-(6-fluoro-4-quinolinyl)-4-(trifluoromethyl)-1H-indazole-1-acetic acid, ethyl ester

NaH (60% dispersion in mineral oil, 22 mg) was added to the product from step c (245 mg) in THF (8 ml) uder nitrogen. The reaction mixture was stirred for 10 min and then ethyl bromoacetate (0.1 ml) was added dropwise and the mixture stirred for a further 1 h. The reaction mixture was quenched with water, extracted (EtOAC). The organic phase was dried (MgSO₄) and concentrated in vacuo. The residue was purified by chomatography (silica, eluting EtOAC:hexame 3:7), to give the subtitle compound (100 mg).

MS ESI+ 346 [M+1]

e) 3-(6-fluoro-4-quinolinyl)-4-(trifluoromethyl)-1H-indazole-1-acetic acid

The title compound was prepared by the method of example 1 part e) using the product of step d).

¹H NMR (DMSO) δ 5.1 (s,2H), 7.14 (dd,1H), 7.57-7.62 (m,3H), 7.62-7.75 (m,1H), 8.051 (d, 1H), 8.18-8.22 (m,1H) and 9.01 (d,1H).

### EXAMPLE 4

### 4-iodo-3-(4-quinolinyl)-1H-indazole-1-acetic acid

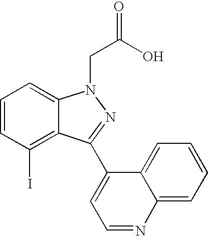

a) α-(2-fluoro-6-iodophenyl)-4-quinolinemethanol

n-BuLi (2.5M) was added dropwise to a stirred solution of diisopropylamine in THF (80 ml) at 0° C. under nitrogen. The reaction mixture was cooled to −78° C. and 1-iodo-3-fluorobenzene (10 g) was added dropwise. The reaction mixture was stirred at this temperature for 1.5 h and the treated with a solution of quinoline-4-aldehyde (7.1 g) in THF (30 ml) and stirred for 10 minutes before quenching with ammonium chloride solution and allowing to reach room temperature. The mixture was diluted with water and ethyl acetate. The organic phase was dried (MgSO4) and concentrated in vacuo. The sub-title compound was obtained as a white solid (6.65 g) after triuration with diethyl ether.

¹H NMR (CDCl₃) δ 2.97-3 (m, 1H), 6.76 (d, 1H), 7-7.5 (m,1H), 7.42 (m, 1H), 7.52-7.58 (m,1H), 7.64-7.79 (m, 2H), 8.02 (d, 1H) and 8.18 (d, 1H).

b) 4-(4-iodo-1H-indazol-3-yl)-quinoline

Prepared using the product of step a) by the methods of example 3 parts b and c.

MS ESI+ 372 [M+1]

c) 4-iodo-3-(4-quinolinyl)-1H-indazole-1-acetic acid, 1,1-dimethylethyl ester

The sub-title compound was prepared by the method of example 1 part d) using the product of part b) (0.82 g) and tertiary-butyl bromoacetate (0.5 ml). The product was used in the next step without any further purification.

d) 4-iodo-3-(4-quinolinyl)-1H-indazole-1-acetic acid

The product of step c) (0.2 g) was dissolved in dichloromethane (4 ml) and treated with TFA (1 ml), stirred overnight at room temperature and concentrated in vacuo. The subtitle compound was further purified by reverse phase HPLC to give the sub-title compound as a yellow solid (93 mg).

¹H NMR (DMSO) δ 5.4 (s,2H), 7.2-7.23 (m,1H), 7.42-7.9 (m,5H), 8.16 (d,1H) and 9.07 (d, 1H).

### EXAMPLE 5

### 3-[(4-chlorophenyl)thio]-5-iodo-1H-indazole-1-acetic acid

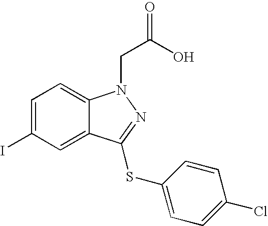

a) 3-[(4-chlorophenyl)thio]-5-iodo-1H-indazole

5-iodoindazole (0.3 g) inDMF (8 ml) was treated with potassium-tertiary-butoxide solution (1.5 ml, 1M in THF) and bis(4-chlorophenyl)disulfide and heated at 65 C for 4 days after which the reaction was quenched with water and extracted with ethyl acetate, dried the organics (MgSO₄) and then concentrated in vacuo. Purified by silica chromatography to afford the product as a white solid.

MS ES+ 387 [M+1]

b) 3-[(4-chlorophenyl)thio]-5-iodo-1H-indazole-1-acetic acid

The sub-title compound was prepared by the methods of example 1 part d) and example 1 part e) using the product from step a).

¹H NMR (CDCl₃) δ 4.98 (s, 2H), 7.17 (dd, 2H), 7.36 (dd,2H), 7.51 (d, 1H), 7.63 (dd,1H) and 7.87 (s,1H)

### EXAMPLE 6

### 3-(7-chloro-4-quinolinyl)-2-methyl-1H-pyrrolo[2,3-b]pyridine-1-acetic acid, sodium salt

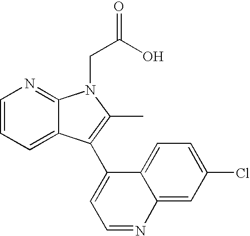

a) 7-chloro-4-(2-methyl-1H-pyrrolo[2,3-b]pyridin-3-yl)-quinoline

2-methyl-1H-pyrrolo[2,3-b]pyridine (0.4 g), 4-chloroquinoline (0.6 g) and N-methyl pyrrolidine (1 ml) were stirred at 100° C. over 2 days. The reaction mixture was triturated with diethyl ether and filtered to give a solid, which was further purified by silica chromatography eluting with ethyl acetate: isoheaxane (3:7) to give the sub-title compound (31 mg).

MS ES+ 293 [M+1]

b) 3-(7-chloro-4-quinolinyl)-1H-pyrrolo[2,3-b]pyridine-1-acetic acid, ethyl ester

The sub-title compound was prepared from the product of step a) by the method of example 1 part d).

MS ES+ 380 [M+1]

c) 3-(7-chloro-4-quinolinyl)-2-methyl-1H-pyrrolo[2,3-b]pyridine-1-acetic acid, sodium salt

The product of part b) (30 mg, 0.09 mmol), sodium hydroxide (0.09 ml), methanol (0.2 ml) and THF (0.2 ml), were stirred at room temperature overnight. The solution was concentrated in vacuo and then triturated with diethyl ether to give the title compound as a whiter solid (20 mg).

¹H NMR (DMSO) δ 2.3 (s, 3H), 4.62 (d, 2H), 7.01-7.06 (M, 1H), 7.5-7.6 (m,3H), 7.8 (d, 1H), 8.15-8.22 (m,2H) and 8.98 (d,1H).

### EXAMPLE 7

### 3-[(4-Chloro-2,4-cyclohexadien-1-yl)thio]-2,5-dimethyl-1H-pyrrolo[3,2-b]pyridine-1-acetic acid

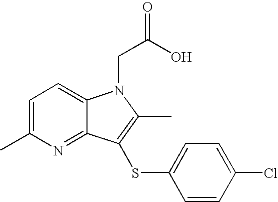

a) 3-[(4-Chlorophenyl)thio]-2,5-dimethyl-1H-pyrrolo[3,2-b]pyridine

A mixture of 2,5-dimethyl-1H-pyrrolo[3,2-b]pyridine (0.5 g) and potassium tert-butoxide (3.7 ml, 1M in tert-butanol) in tert-butanol (25 ml) was heated under reflux for 20 min then bis(4-chlorophenyl)disulphide (1.44 g) added. After heating for a further 1 h, the mixture was cooled and water (100 ml) added. The precipitate was filtered off, washed with water, diethylether and dried to give the title compound (930 mg).

MS ESI+ 289 [M+1]

b) 3-[(4-Chlorophenyl)thio]-2,5-dimethyl-1H-pyrrolo[3,2-b]pyridine-1-acetic acid, ethyl ester

The subtitle compound was prepared by the method of example 1 part d, using the product of step a).

MS ESI+ 375 [M+1]

c) 3-[(4-Chloro-2,4-cyclohexadien-1-yl)thio]-2,5-dimethyl-1H-pyrrolo[3,2-b] pyridine-1-acetic acid

The product from step b (355 mg) was treated with aqueous NaOH (1M, 0.95 ml), THF (15 ml) and water (2 ml). The resulting solution was stirred at room temperature for 5 h then evaporated under reduced pressure. The residue was triturated with ether, filtered and dried to give the title compound (0.302 g)

δ_{H} (DMSO) 2.39 (s,3H), 2.47 (s,3H), 4.46 (s,2H), 6.69-7.25 (m,5H), 7.65 (d,1H).

MS APCI− 345 [M−1]

### EXAMPLE 8

### 2,5-Dimethyl-3-[[4-(methylsulfonyl)-2,4-cyclohexadien-1-yl]methyl]-1H-pyrrolo[3,2-b]pyridine-1-acetic acid

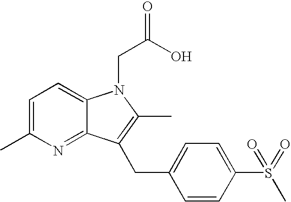

a) 2,5-Dimethyl-3-[[4-(methylsulfonyl)phenyl]methyl]-1H-pyrrolo[3,2-b]pyridine

Ethylmagnesium bromide (1.2 ml, 3M in diethylether) was added to a stirred solution of 2,5-dimethyl-1H-pyrrolo[3,2-b]pyridine (0.44 g) in THF (20 ml) at RT. After 30 min p-methylsulfonylbenzyl bromide (0.7 g) was added and the mixture heated under reflux for 2 h. DMF (5 ml) was added, the mixture heated under reflux for 4 h, cooled and partitioned between ethylacetate and water. The organic layer was separated, washed with water, dried and evaporated under reduced pressure. The residue was purified by chromatography on silica eluting with 5% methanol/DCM to give the title compound (0.121 g)

MS ESI+ 315 [M+1]

b) 2,5-Dimethyl-3-[[4-(methylsulfonyl)phenyl]methyl]-1H-pyrrolo[3,2-b]pyridine-1-acetic acid, ethyl ester

The product from step a) (0.115 g), potassium carbonate (0.2 g) and ethylbromoacetate (0.05 ml) in DMF (5 ml) were heated at 50° C. for 5 h. Ethyl bromoacetate (0.1 ml) was added and the mixture heated for a further 4 h at 80° C., cooled then partitioned between diethylether and water. The organic layer was separated, washed with water, dried and evaporated under reduced pressure. The residue was purified by chromatography on silica eluting with 2-3% methanol/DCM to give the sub-title compound (73 mg).

MS ESI+ 401 [M+1]

c) 2,5-Dimethyl-3-[[4-(methylsulfonyl)-2,4-cyclohexadien-1-yl]methyl]-1H-pyrrolo[3,2-b]pyridine-1-acetic acid

The product from step b) (72 mg) was treated with aqueous NaOH (1M, 0.3 ml), THF (5 ml) and water (3 ml). The resulting solution was stirred at room temperature for 3 h then 2M HCl (3 ml) added. The aqueous layer was extracted with ethylacetate then evaporated under reduced pressure. The residue was recrystallised from water to give the sub-title compound (40 mg).

¹H NMR (DMSO) δ 2.40 (s,3H), 2.81 (s,3H), 3.17 (s,3H), 4.52 (s,2H), 5.27 (s,2H), 7.45 (d,1H), 7.49 (d,2H), 7.82 (d,2H), 8.57 (d,1H), 15.74 (s,1H).

MS APCI− 371 [M−1]

### EXAMPLE 9

### 2,5-Dimethyl-3-[[4-(methylsulfonyl)phenyl]thio]-1H-pyrrolo[3,2-b]pyridine-1-acetic acid

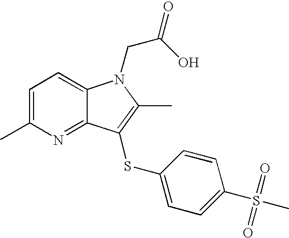

a) 2,5-Dimethyl-3-[[4-(methylsulfonyl)phenyl]thio]-1H-pyrrolo[3,2-b]pyridine

A mixture of 2,5-dimethyl-1H-pyrrolo[3,2-b]pyridine (0.4 g), potassium carbonate (0.7 g) and bis(4-methylsulphonylphenyl)disulphide (1.2 g) in DMF (15 ml) were stirred at RT for 4 days then heated at 50° C. for 6 h. The mixture was partitioned between ethylacetate and water, the organic layer was separated, washed with water, dried and evaporated under reduced pressure. The residue was triturated with ethylacetate and filtered to give the sub-title compound (0.3 g)

MS ESI+ 333 [M+1]

b) 2,5-Dimethyl-3-[[4-(methylsulfonyl)phenyl]thio]-1H-pyrrolo[3,2-b]pyridine-1-acetic acid, 1,1-dimethylethyl ester

The product from step a) (0.3 g), potassium carbonate (0.3 g) and tert-butylbromoacetate (0.13 ml) in DMF (8 ml) were stirred at RT for 18 h. The mixture was partitioned between ethylacetate and water, the organic layer separated, washed with water, dried and evaporated under reduced pressure. The residue was triturated with ethylacetate/isohexane to give the sub-title compound (0.175 g)

MS ESI+ 447 [M+1]

c) 2,5-Dimethyl-3-[[4-(methylsulfonyl)phenyl]thio]-1H-pyrrolo[3,2-b]pyridine-1-acetic acid

The product from step b) (0.175 g), trifluoracetic acid (5 ml) and DCM (10 ml) were stirred at RT for 16 h then evaporated under reduced pressure. The residue was triturated with diethylether and filtered to give the title compound (125 mg).

¹H NMR (DMSO) δ 2.71 (s,3H), 3.16 (s,3H), 5.34 (s,2H), 7.21 (d,2H), 7.43 (brd,1H), 7.75 (d,2H), 8.46 (brs,1H).

MS APCI− 389 [M−1]

### EXAMPLE 10

### 4-Chloro-3-[(4-chlorophenyl)thio]-2-methyl-1H-pyrrolo[3,2-c]pyridine-1-acetic acid

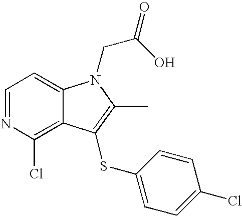

a) [2-Chloro-3-(1-propynyl)-4-pyridinyl]-carbamic acid, 1,1-dimethylethyl ester

A mixture of (2-chloro-3-iodo-pyridin-4-yl)-carbamic acid tert-butyl ester (3.4 g), copper(I) iodide (0.09 g), triethylamine (2.8 ml), propyne (approx. 1 g) and dichloro(bistriphenylphosphine)palladium (0.2 g) in DMF (30 ml) was heated at 50° C. for 5 h. The mixture was partitioned between diethylether and water, the organics separated, washed with water, dried and evaporated under reduced pressure. The residue was purified by chromatography on silica eluting with 10% ethylacetate/isohexane to give the sub-title compound (2.14 g).

MS ESI− 265/7 [M−1]

b) 4-Chloro-2-methyl-1H-pyrrolo[3,2-c]pyridine

The product from step a) (2.1 g) and copper(I) iodide (0.035 g) in DMF (50 ml) was heated at 90° C. for 6 h, cooled and partitioned between ethylacetate and brine. The organics were separated, washed with brine, dried and evaporated under reduced pressure. The residue was purified by chromatography on silica eluting with 40% ethylacetate/isohexane to give the sub-title compound (0.785 g).

¹H NMR (DMSO) δ 2.42 (s,3H), 6.24 (s,1H), 7.29 (d,1H), 7.87 (d,1H), 11.76 (s,1H).

c) 4-Chloro-3-[(4-chlorophenyl)thio]-2-methyl-1H-pyrrolo[3,2-c]pyridine

The subtitle compound was prepared by the method of example 9 part a), using the product of step b).

MS ESI+ 309/11 [M+1]

d) 4-Chloro-3-[(4-chlorophenyl)thio]-2-methyl-1H-pyrrolo[3,2-c]pyridine-1-acetic acid, 1,1-dimethylethyl ester

The subtitle compound was prepared by the method of example 9 part b), using the product of step c).

MS ESI+ 423/5 [M+1]

e) 4-Chloro-3-[(4-chlorophenyl)thio]-2-methyl-1H-pyrrolo[3,2-c]pyridine-1-acetic acid

The title compound was prepared by the method of example 9 part c), using the product of step d).

¹H NMR (DMSO) δ 2.46 (s,3H), 5.23 (s,2H), 6.98 (d,2H), 7.29 (d,2H), 7.69 (d,1H), 8.05 (d,1H).

MS APCI− 365/7 [M−1]

### EXAMPLE 11

### 4-Chloro-2-methyl-3-[[4-(methylsulfonyl)phenyl]thio]-1H-pyrrolo[3,2-c]pyridine-1-acetic acid

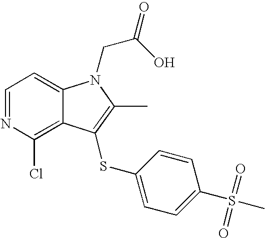

The title compound was prepared by the method of example 10.

¹H NMR (DMSO) δ 2.71 (s,3H), 3.16 (s,3H), 5.34 (s,2H), 7.21 (d,2H), 7.43 (brd,1H), 7.75 (d,2H), 8.46 (brs,1H).

MS APCI− 389 [M-−b 1]

### EXAMPLE 12

### 3-[(4-Chlorophenyl)thio]-2-methyl-4-phenyl-1H-pyrrolo[3,2-c]pyridine-1-acetic acid

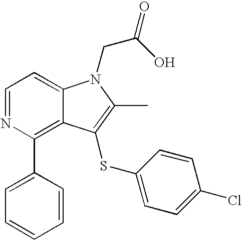

a) 4-Chloro-2-methyl-1H-pyrrolo[3,2-c]pyridine-1-carboxylic acid, 1,1-dimethylethyl ester

The product from example 10 part b) (0.5 g), di-tert-butyl dicarbonate (0.655 g) and 4-dimethyl aminopyridine (0.05 g) in DCM (20 ml) was stirred at RT for 72 h then partitioned between diethylether and water. The organics were separated, washed with water, dried and evaporated under reduced pressure to give the sub-title compound (0.8 g).

MS ESI+ 267/9 [N+1]

b) 2-Methyl-4-phenyl-1H-pyrrolo[3,2-c]pyridine-1-carboxylic acid, 1,1-dimethylethyl ester

The product from step a) (0.6 g), cesium fluoride (0.87 g), phenylboronic acid (0.45 g) and tetrakis(triphenylphosphine)palladium(0) (0.1 g) in dioxane (20 ml) were heated at 100° C. for 5 h, cooled and partitioned between diethylether and water. The organics were separated, washed with water, dried and evaporated under reduced pressure. The residue was purified by chromatography on silica eluting with 20% ethylacetate/isohexane to give the sub-title compound (0.627 g).

MS ESI+ 309 [M+1]

c) 2-Methyl-4-phenyl-1H-pyrrolo[3,2-c]pyridine, trifluoroacetate salt

The product from step b) (0.62 g), trifluoroacetic acid (5 ml) and DCM (15 ml) were stirred at RT for 24 h then evaporated under reduced pressure. Used crude in next step.

d) 3-[(4-Chlorophenyl)thio]-2-methyl-4-phenyl-1H-pyrrolo[3,2-c]pyridine

The subtitle compound was prepared by the method of example 9 part a), using the product of step c).

MS ESI+ 351/3 [M+1]

e) 3-[(4-Chlorophenyl)thio]-2-methyl-4-phenyl-1H-pyrrolo[3,2-c]pyridine-1-acetic acid, 1,1-dimethylethyl ester

The subtitle compound was prepared by the method of example 9 part b), using the product of step d).

MS ESI+ 465/7 [M+1]

f) 3-[(4-Chlorophenyl)thio]-2-methyl-4-phenyl-1H-pyrrolo[3,2-c]pyridine-1-acetic acid

The title compound was prepared by the method of example 9 part c), using the product of step e).

¹H NMR (DMSO) δ 2.53 (s,3H), 5.47 (s,2H), 6.58 (d,2H), 7.14 (d,2H), 7.35-7.56 (m,5H), 8.28 (d,1H), 8.55 (d,1H).

MS APCI− 407/9 [M−1]

### EXAMPLE 13

### 2-Methyl-3-[[4-(methylsulfonyl)phenyl]thio]-4-phenyl-1H-pyrrolo[3,2-c] pyridine-1-acetic acid

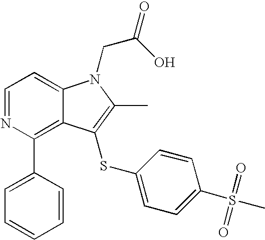

The title compound was prepared by the method of example 11.

¹H NMR (DMSO) δ 2.53 (s,3H), 3.15 (s,3H), 5.49 (s,2H), 6.80 (d,2H), 7.29-7.51 (m,5H), 7.57 (d,2H), 8.30 (d,1H), 8.57 (d,1H).

MS APCI− 451 [M−1]

### EXAMPLE 14

### 3-(7-chloro-4-quinolinyl)-2,3-dihydro-5-methyl-2-oxo-1H-benzimidazole-1-acetic acid

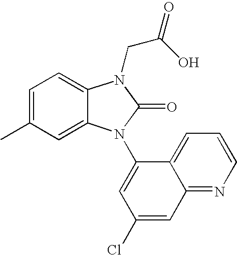

a) 2-[(7-chloro-4-quinolinyl)amino]-4-methyl-benzoic acid

A solution of 2-amino-4-methylbenzoic acid (2 g) and 4,7-dichloroquinoline (2.62 g) in NMP was stirred at 140° C. for 4 hours. The reaction mixture was cooled to room temperature and added to brine to afford a precipitate which was filtered, washed with water and dried to afford the sub-title compound (4.04 g).

MS ES+ 313 [M+1]

b) 1-(7-chloro-4-quinolinyl)-1,3-dihydro-6-methyl-2H-benzimidazol-2-one

A suspension of the product of part a) (2 g) in dry DMF was treated with triethylamine (0.9 ml) and stirred for 30 min. Diphenylphosphoryl azide (1.38 ml) was added and the reaction stirred for a further 2 hours, then at 60° C. for 4 hours. The reaction was cooled to room temperature and added to brine and the suspension extracted with ethyl acetate. The combined organic extracts were dried (MgSO₄) and concentrated in vacuo. The resulting solid was triturated and dried to give the sub-title compound (1.06 g).

3-(7-chloro-4-quinolinyl)-2,3-dihydro-5-methyl-2-oxo-1H-benzimidazole-1-acetic acid

The sub-title compound was prepared by the methods of example 1 parts d) and e).

¹H NMR (DMSO) δ 2.23 (s,3H), 4.74 (d,2H), 6.54 (s,1H), 7.07 (d, 1H), 7.25 (d,1H), 7.64 (m,2H), 7.77 (d,1H), 8.28 (s,1H), 9.15 (d, 1H) and 13.22 (s, 1H).

MS APCI+ 369 [M+1]

### Pharmacological Data

### Ligand Binding Assay

[³H]PGD₂ was purchased from Perkin Elmer Life Sciences with a specific activity of 100-210 Ci/mmol. All other chemicals were of analytical grade.

HEK cells expressing rhCRTh2/Gα16 were routinely maintained in DMEM containing 10% Foetal Bovine Serum (HyClone), 1 mg/ml geneticin, 2 mM L-glutamine and 1% non-essential amino acids. For the preparation of membranes, the adherent transfected HEKcells were grown to confluence in two layer tissue culture factories (Fisher, catalogue number TKT-170-070E). Maximal levels of receptor expression were induced by addition of 500 mM sodium butyrate for the last 18 hours of culture. The adherent cells were washed once with phosphate buffered saline (PBS, 50 ml per cell factory) and detached by the addition of 50 ml per cell factory of ice-cold membrane homogenisation buffer [20 mM HEPES (pH 7.4), 0.1 mM dithiothreitol, 1 mM EDTA, 0.1 mM phenyl methyl sulphonyl fluoride and 100 μg/ml bacitracin]. Cells were pelleted by centrifugation at 220×g for 10 minutes at 4° C., re-suspended in half the original volume of fresh membrane homogenisation buffer and disrupted using a Polytron homogeniser for 2×20 second bursts keeping the tube in ice at all times. Unbroken cells were removed by centrifugation at 220×g for 10 minutes at 4° C. and the membrane fraction pelleted by centrifugation at 90000×g for 30 minutes at 4° C. The final pellet was re-suspended in 4 ml of membrane homogenisation buffer per cell factory used and the protein content determined. Membranes were stored at −80° C. in suitable aliquots.

All assays were performed in Corning clear bottomed, white 96-well NBS plates (Fisher). Prior to assay, the HEK cells membranes containing CRTh2 were coated onto SPA PVT WGA beads (Amersham). For coating membranes were incubated with beads at typically 25 μg membrane protein per mg beads at 4° C. with constant agitation overnight. (The optimum coating concentrations were determined for each batch of membranes) The beads were pelleted by centrifugation (800×g for 7 minutes at 4° C.), washed once with assay buffer (50 mM HEPES pH 7.4 containing 5 mM magnesium chloride) and finally re-suspended in assay buffer at a bead concentration of 10 mg/ml.

Each assay contained 20 μl of 6.25 nM [³H]PGD₂, 20 μl membrane saturated SPA beads both in assay buffer and 10 μl of compound solution or 13,14-dihydro-15-keto prostaglandin D₂ (DK-PGD₂, for determination of non-specific binding, Cayman chemical company).

Compounds and DK-PGD₂ were dissolved in DMSO and diluted in the same solvent to 100× the required final concentration. Assay buffer was added to give a final concentration of 10% DMSO (compounds were now at 10× the required final concentration) and this was the solution added to the assay plate. The assay plate was incubated at room temperature for 2 hours and counted on a Wallac Microbeta liquid scintillation counter (1 minute per well).

Compounds of formula (I) have an IC₅₀ value of less than (<) 10 μM.

1. A compound of formula (I) or a pharmaceutically acceptable salt thereof: 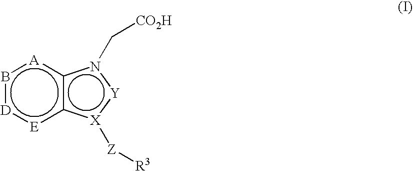
in which
each of A, B, D and E is independently C—R¹ or N;
Y═C—R², N or C═O;
Z is oxygen, sulphur, a C₁₋₆alkylene chain or a bond;
R¹ is independently selected from hydrogen, halogen, CN, nitro, S(O)ₓR⁶, OR⁶, SO₂NR⁴R⁵, CONR⁴R⁵, NR⁴R⁵, NR⁷SO₂R⁷, NR⁷C(O)ₓR⁷, C₂-C₆ alkenyl, C₂-C₆ alkynyl, C₁₋₆alkyl, aryl or heteoroaryl, the latter five groups being optionally substituted by one or more substituents independently selected from 1-3 halogen atoms, —OR⁷ and —NR⁴R⁵, S(O)xR⁸, C(O)NR⁴R⁵, where x is 0,1 or 2;
R² is C₁₋₆alkyl which may be optionally substituted by one or more substituents independently selected from halogen atoms, aryl, —OR⁹ and —NR¹⁰R¹¹;
R³ is an aryl or heteroaryl group each of which is optionally substituted by one or more substituents independently selected from halogen, CN, nitro, S(O)ₓR⁶, OR⁷, SO₂NR⁴R⁵, CONR⁴R⁵, NR⁴R⁵, NR⁷SO₂R³, NR⁷C(O)ₓR⁶, C₂-C₆ alkenyl, C₂-C₆ alkynyl, C₁₋₆ alkyl, the latter three groups being optionally substituted by one or more substituents independently selected from halogen atoms, —OR⁶ and —NR⁴R⁵, where x=0,1 or 2;
R⁴ and R⁵ independently represent a hydrogen atom, a C₁₋₆alkyl group, or aryl group the latter two of which may be optionally substituted by one or more substituent groups independently selected from halogen atoms, aryl, —OR¹² and —NR¹³R¹⁴, —CONR¹³R¹⁴, —NR¹³COR¹⁴, —SO₂NR¹³R¹⁴, NR¹³SO₂R¹⁴; or
R⁴ and R⁵ together with the nitrogen atom to which they are attached can form a 3-8 membered saturated heterocylic ring optionally containing one or more atoms selected from O, S, NR¹⁵, and itself optionally substituted by C₁₋₃ alkyl, halogen;
R⁶ represents a C₁₋₆alkyl which may be optionally substituted by one or more substituents independently selected from halogen atoms, aryl, —OR⁹ and —NR¹⁰R¹¹.
each of R⁷, R⁸ R⁹, R¹⁰, R¹¹, R¹², R¹³, R¹⁴, independently represents a hydrogen atom, C₁-C₆, alkyl, an aryl or a heteroaryl group which may be optionally substituted by one or more halogen atoms, OH, O—C₁-C₆alkyl; and
R¹⁵ is hydrogen, C₁₋₄ alkyl, —COC₁-C₄ alkyl, —COQC₁-C₄alkyl, Q=O or NR⁶,
provided that:
the number of nitrogen atoms within the ring ABDE is 1 or 2 when Y is CR² and R³ cannot be phenyl when Y is C═O and X is nitrogen.

2. A compound according to claim 1 in which A, B, D and E are all C—R¹.

3. A compound according to claim 1 in which one of A, D or E is N and D and the others are C—R¹ where R¹ is hydrogen, phenyl, CF₃, CN, alkyl or halogen.

4. A compound according to claim 1 in which Y is C═O and X is N.

5. compound according to claim 4 in which Z is a bond.

6. A compound according to claim 1 in which Y is nitrogen or C—R² where R is methyl.

7. A compound according to claim 6 in which X is carbon,

8. A compound according to claim 1 in which Z is sulfur, methylene or a bond.

9. A compound according to claim 1 selected from:
5-methyl-3-(4-quinolinyl)-1H-indazole-1-acetic acid;
5-cyano-3-(4-quinolinyl)-1H-indazole-1-acetic acid;
3-(6-fluoro-4-quinolinyl)-4-(trifluoromethyl)-1H-indazole-1-acetic acid;
4-iodo-3-(4-quinolinyl)-1H-indazole-1-acetic acid;
3-[(4-chlorophenyl)thio]-5-iodo-1H-indazole-1-acetic acid;
3-(7-chloro-4-quinolinyl)-2-methyl-1H-pyrrolo[2,3-b]pyridine-1-acetic acid, sodium salt;
3-[(4-Chloro-2,4-cyclohexadien-1-yl)thio]-2,5-dimethyl-1H-pyrrolo[3,2-b]pyridine-1-acetic acid;
2,5-Dimethyl-3-[[4-(methylsulfonyl)-2,4-cyclohexadien-1-yl]methyl]-1H-pyrrolo[3,2-b]pyridine-1-acetic acid;
2,5-Dimethyl-3-[[4-(methylsulfonyl)phenyl]thio]-1H-pyrrolo[3,2-b]pyridine-1-acetic acid;
4-Chloro-3-[(4-chlorophenyl)thio]-2-methyl-1H-pyrrolo[3,2-c]pyridine-1-acetic acid;
4-Chloro-2-methyl-3-[[4-(methylsulfonyl)phenyl]thio]-1H-pyrrolo[3,2-c]pyridine-1-acetic acid;
3-[(4-Chlorophenyl)thio]-2-methyl-4-phenyl-1H-pyrrolo[3,2-c]pyridine-1-acetic acid;
2-Methyl-3-[[4-(methylsulfonyl)phenyl]thio]-4-phenyl-1H-pyrrolo[3,2-c] pyridine-1-acetic acid;
and pharmaceutically acceptable salts thereof.

10. (canceled)

11. A method of treating a disease mediated by prostaglandin D2, which comprises administering to a patient a therapeutically effective amount of a compound of formula (I), or a pharmaceutically acceptable salt as defined in claim 1.

12. A method of treating according to claim 11 wherein the disease is asthma or rhinitis.

13. (canceled)

14. The method according to claim 11 wherein the disease is asthma or rhinitis.

15. The method according to claim 11 wherein the compound is selected from:
5-methyl-3-(4-quinolinyl)-1H-indazole-1-acetic acid;
5-cyano-3-(4-quinolinyl)-1H-indazole-1-acetic acid;
3-(6-fluoro-4-quinolinyl)-4-(trifluoromethyl)-1H-indazole-1-acetic acid;
4-iodo-3-(4-quinolinyl)-1H-indazole-1-acetic acid;
3-[(4-chlorophenyl)thio]-5-iodo-1H-indazole-1-acetic acid;
3-(7-chloro-4-quinolinyl)-2-methyl-1H-pyrrolo[2,3-b]pyridine-1-acetic acid, sodium salt;
3-[(4-Chloro-2,4-cyclohexadien-1-yl)thio]-2,5-dimethyl-1H-pyrrolo[3,2-b]pyridine-1-acetic acid;
2,5-Dimethyl-3-[[4-(methylsulfonyl)-2,4-cyclohexadien-1-yl]methyl]-1H-pyrrolo[3,2-b]pyridine-1-acetic acid;
2,5-Dimethyl-3-[[4-(methylsulfonyl)phenyl]thio]-1H-pyrrolo[3,2-b]pyridine-1-acetic acid;
4-Chloro-3-[(4-chlorophenyl)thio]-2-methyl-1H-pyrrolo[3,2-c]pyridine-1-acetic acid;
4-Chloro-2-methyl-3-[[4-(methylsulfonyl)phenyl]thio]-1H-pyrrolo[3,2-c]pyridine-1-acetic acid;
3-[(4-Chlorophenyl)thio]-2-methyl-4-phenyl-1H-pyrrolo[3,2-c]pyridine-1-acetic acid;
2-Methyl-3-[[4-(methylsulfonyl)phenyl]thio]-4-phenyl-1H-pyrrolo[3,2-c] pyridine-1-acetic acid;
and pharmaceutically acceptable salts thereof.
